(19) United States
(12) Patent Application Publication
(10) Pub. No.: US 2025/0305762 A1
(43) Pub. Date: Oct. 2, 2025

(54) INTELLIGENT CRISPER

(71) Applicant: Whirlpool Corporation, Benton Harbor, MI (US)

(72) Inventors: Alison W. Pereira, St. Joseph, MI (US); Leann N. Vaive, St. Joseph, MI (US); Matheus Zappelini, Joinville (BR); Morgana Minich, Joinville (BR); Jakub Brzezina, WROCLAW (PL); Marcin Nieslony, WROCLAW (PL); Mario Togawa, Monterrey (MX)

(21) Appl. No.: 19/062,826

(22) Filed: Feb. 25, 2025

Related U.S. Application Data
(60) Provisional application No. 63/570,981, filed on Mar. 28, 2024.

(51) Int. Cl. F25D 29/00; F25D 21/00

(52) U.S. Cl. CPC F25D 29/008; F25D 21/008; F25D 2400/36; F25D 2700/02

(57) ABSTRACT

Spoiled food detection by a refrigerator is performed. Gas sensor data is received from a gas sensor, the gas sensor data being indicative of detectable gases emitted from food items stored in a food compartment. Responsive to completion of an air replacement operation in the food compartment, the gas sensor data is used to determine whether spoiled food items are detected in the food compartment. An indication of a level of spoilage of food items detected in the food compartment is displayed to a user interface.

### CROSS-REFERENCE TO RELATED APPLICATIONS

This application claims the benefit of U.S. provisional application Ser. No. 63/570,981 filed Mar. 28, 2024, the disclosure of which is hereby incorporated in its entirety by reference herein.

### TECHNICAL FIELD

Aspects of the present disclosure generally relate to an intelligent food compartment, and more specifically to a food compartment using a gas sensor to evaluate the level of ripeness of food items located within the food compartment.

### BACKGROUND

Appliances, such as refrigeration appliances, conventionally include various shelves and drawers within the cabinet area of the appliance. Food items ripen slower in the refrigerator as compared to at room temperature. Depending on the food, for temperatures above freezing, the colder the storage, the slower the ripening. Thus, storing food items in the refrigerator will extend their life. Either way, once fully ripened food items should be consumed or discarded. When consumers store food in the fridge they do not always consume it quickly. Thus, many times the consumer has to throw away the food because it has spoiled.

### SUMMARY

In one or more examples, a method for performing spoiled food detection by a refrigerator is performed. The method includes receiving gas sensor data from a gas sensor, the gas sensor data being indicative of detectable gases emitted from food items stored in a food compartment; responsive to completion of an air replacement operation in the food compartment, using the gas sensor data to determine whether spoiled food items are detected in the food compartment; and displaying, to a user interface, an indication of a level of spoilage of food items detected in the food compartment.

In one or more examples, the air replacement operation is a defrost cycle.

In one or more examples, the method includes scheduling the defrost cycle; and checking for presence of spoiled food items based on the gas sensor data, responsive to completion of the scheduled defrost cycle.

In one or more examples, the defrost cycle is scheduled periodically.

In one or more examples, the air replacement operation includes a predefined amount of door openings.

In one or more examples, the method includes recording a history of operation of a door to the refrigerator, the history indicating timing of openings and closings of the door.

In one or more examples, the method includes, responsive to the history indicating at least the predefined amount of door openings open since a last defrost cycle, inhibiting a next defrost cycle.

In one or more examples, the method includes checking for presence of spoiled food items based on the gas sensor data, despite the inhibiting of the next defrost cycle, due to the door openings clearing air in the food compartment to facilitate measurement of the emitted gases.

In one or more examples, the amount of door openings includes a total amount of time that the door is open.

In one or more examples, the amount of door openings includes a quantity of times that the door is opened.

In one or more examples, the gas sensor is embedded in a shelf frame housing.

In one or more examples, the shelf frame housing further includes the user interface.

In one or more examples, a refrigerator implementing spoiled food detection includes a shelf frame sensor housing comprising a user interface and a gas sensor configured to provide gas sensor data indicative of gases emitted from food items stored in the refrigerator; and
one or more controllers configured to: responsive to completion of an air replacement operation in the refrigerator, use a gas signal from the gas sensor to determine whether spoiled food items are detected in the refrigerator, and display, to the user interface, an indication of a level of spoilage of food items detected in the refrigerator.

In one or more examples, the air replacement operation is a defrost cycle performed by the refrigerator.

In one or more examples, the one or more controllers are further configured to schedule the defrost cycle; and check for presence of spoiled food items based on the gas signal, responsive to completion of the scheduled defrost cycle.

In one or more examples, the one or more controllers are further configured to schedule the defrost cycle on a periodic timeframe.

In one or more examples, the air replacement operation includes a predefined amount of door openings.

In one or more examples, the one or more controllers are further configured to record a history of operation of a door to the refrigerator, the history indicating timing of openings and closings of the door.

In one or more examples, the one or more controllers are further configured to responsive to the history indicating at least the predefined amount of door openings open since a last defrost cycle, inhibit a next defrost cycle.

In one or more examples, the one or more controllers are further configured to check for presence of spoiled food items based on the gas sensor data, despite the inhibiting of the next defrost cycle, due to the door openings clearing air in the refrigerator to facilitate measurement of the emitted gases.

In one or more examples, the amount of door openings includes a total amount of time that the door is open.

In one or more examples, the amount of door openings includes a quantity of times that the door is opened.

In one or more examples, the gas sensor is embedded in a shelf frame housing of a shelf of the refrigerator.

In one or more examples, the shelf frame housing further includes the user interface.

### BRIEF DESCRIPTION OF THE DRAWINGS

FIG. 1 is a schematic illustration of an appliance implementing an intelligent food compartment feature, with doors in a closed position, according to one or more embodiments;

FIG. 2 shows the appliance of FIG. 1, with the doors in an open position;

FIG. 3 illustrates a schematic view of operation of a controller of the refrigerator of FIG. 1;

FIG. 4 illustrates a graph of sensor data received from the gas sensor over time in view of a defrost cycle;

FIG. 5 illustrates a graph of sensor data received from the gas sensor over time in view of a door opening;

FIG. 6 illustrates an example exploded perspective view of components of the shelf frame sensor housing of the refrigerator of FIG. 1;

FIG. 7 illustrates a cutaway perspective view of the shelf frame sensor housing of FIG. 6 in an assembled state, showing the placement of certain internal components;

FIG. 8 illustrates a close-up perspective view detailing the crisper user interface and the wire connection of the shelf frame sensor housing of FIG. 6;

FIG. 9 a close-up perspective view of the wire connection of the shelf frame sensor housing of FIG. 6 in a covered state;

FIG. 10 illustrates an example process for the operation of the refrigerator of FIG. 1 implementing the intelligent crisper feature; and

FIG. 11 illustrates an example of a computing device for implementing the intelligent crisper feature.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications.

A refrigerator may use a sensor that measures a level of gas in the food compartment. The gas may be one of various volatile compounds such as trimethylamine, fatty acids, biogenic amines, alcohols, ammonia, etc., whose presence is indicative of food ripening. The refrigerator may apply the sensor data to an algorithm that evaluates a level of ripeness of food items based on the gas level.

Such systems may be sensitive to noise in the sensor data. Sources of noise may include perfumes, cleaning products, or other airborne emissions that may be detectable by the gas sensor. As another source of inaccuracy, gas may remain present in the refrigerator even after a ripe food item has been removed from the refrigerator. Thus, the presence of perfume, cleaning products, or lingering food gases may cause the ripeness determination to produce incorrect results.

To address these issues, the air in the food compartment of the refrigerator may be at least partially replaced, prior to the gas sensor being used to collect the data to be applied to the ripeness algorithm. This may allow for the decision-making to be performed with greater accuracy.

The air cycling may be performed in various ways. In one example, the air cycling may be performed by a defrost cycle. In general, the defrost cycle may be responsible for melting any existing frost that may have formed around the evaporator tubes or other components of the cooling system, eliminating this humidity from the system. This is done to maintain cooling performance. Responsive to the refrigerator detecting a need for a defrost, the compressor and the evaporator fan are turned off, a defrost heater is activated and the heater is powered for a predefined period of time. In some designs, the defrost cycle is scheduled periodically, such as on the order of hourly or every other hour. Responsive to completion of the defrost cycle, the compressor and fan are turned on again, which may serve to trap any remaining cabinet humidity back to the evaporator tubes.

Expressly calling a defrost before measuring data to be applied to the ripeness algorithm helps reset the system, eliminating the remaining gases before making any analysis. However, forcing a defrost may cause unnecessary power consumption and/or cooling issues with the refrigerator.

As the defrost cycle runs periodically, the ripeness algorithm may take advantage of the existing timing of the defrost cycle to periodically perform the ripeness determination upon completion of the defrost cycle. Thus, the existing air cycling inherent in the defrost cycle may be used to allow the ripeness determination to be accurately performed without requiring additional defrost cycles.

Moreover, the air cycling may also be performed by a user opening the refrigerator. Similar to the defrost cycle, door openings may be highly effective in renewing the air inside the crisper. By tracking door openings performed by the user, the ripeness determination may be performed after the user has manually renewed the air in the food compartment. This may allow for the ripeness determination to be accurately performed, even if a defrost cycle has not recently been performed. Moreover, this detection of the door opening may also be used to inhibit the next defrost cycle, if the door openings indicate that sufficient air replacement has already taken place.

In some implementations, the gas sensor and a user interface may be integrated into an intelligent crisper drawer. In an example, an enhanced shelf frame may be used to retain the sensor and user interface elements in proximity to the intelligent crisper drawer. In such an example, the sensor may capture the gases coming from any food items stored in the crisper drawer. Using the ripeness algorithm, the user interface may inform the consumer about the current ripeness of the food items in the crisper compartment. Further aspects of the disclosure are discussed in detail herein.

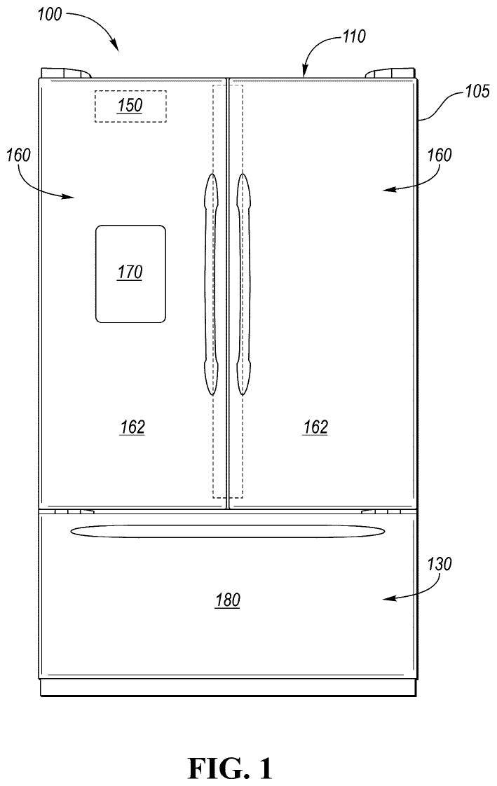

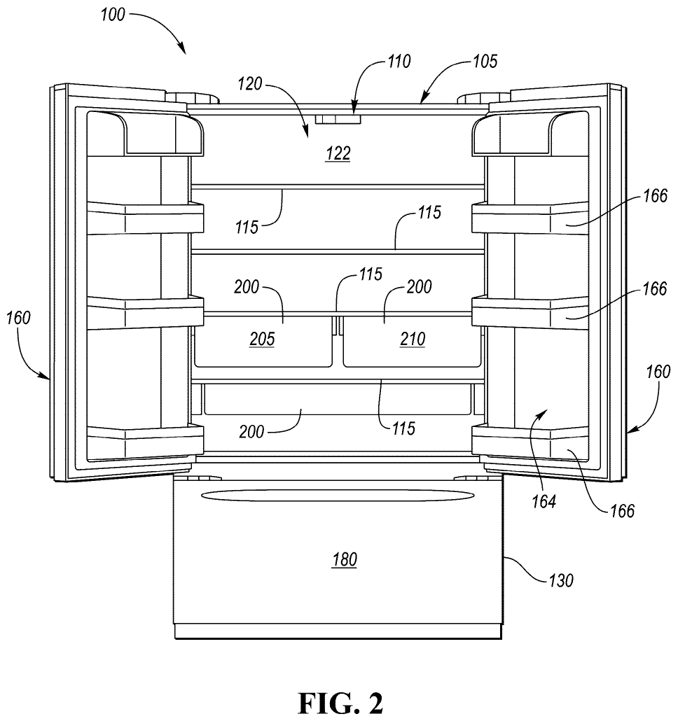

Referring to FIGS. 1 and 2, an appliance 100 is shown according to an embodiment. The appliance 100 may be a refrigeration appliance as shown, however, appliance 100 may be any suitable appliance having compartments for food storage, such as, but not limited to, a refrigerator, freezer, deep freeze, etc. Thus, the depiction or discussion of a refrigerator is not intended to be limiting, and appliance 100 may be referred to interchangeably with refrigerator 100. The appliance 100 of FIGS. 1 & 2 is generally shown as a French-Door Bottom Mount appliance. However, it should be understood that this is not intended to be limiting, and the appliance 100 may be any construction for a refrigerator and/or freezer appliance, such as a side-by-side, two-door bottom mount, or a top-mount type.

As shown in FIGS. 1 and 2, the appliance 100 includes external walls 105 defining a housing 110 with an fresh food compartment 120 formed therein as a first internal storage chamber, internal cavity, or interior cabinet (hereinafter used interchangeably) for refrigerating, and not freezing, consumables or foodstuffs stored within the fresh food compartment 120. The external walls 105 and housing 110 further define a freezer compartment 130 (under the fresh food compartment 120 in the example of FIGS. 1 & 2 showing a bottom-mount French Door construction), as a second internal storage chamber for freezing consumables or foodstuffs within the freezer compartment 130 during normal use. It is generally known that the freezer compartment 130 is typically kept at a temperature below the freezing point of water, and the fresh food compartment 120 is typically kept at a temperature above the freezing point of water and generally below a temperature of from about 35° F. to about 50° F., more typically below about 38° F. Although not shown in the Figures, the refrigerator 100 may include a third pull-out compartment or any additional compartments. The compartments may be separate compartments within narrow cabinet sections or separate cabinet sections or sub-compartments accessible by opening an access door, for example, to access the interior volume of the fresh food compartment 120. Thus, any configuration of a refrigerator/freezer combination or any other multiple zone refrigeration device is contemplated.

The fresh food compartment 120 of the refrigerator 100 is defined by internal walls 122 that form the fresh food compartment 120 and the freezer compartment 130. The internal walls 122 may more specifically form an internal liner of the refrigerator 100. The internal walls 122 may include a rear or back wall, a top wall, a bottom wall, and two side walls, and may be formed of a liner system with insulation between the liner system and the external walls 105 for the appliance 100.

As shown in FIG. 2, the appliance 100 includes one or more shelves 115 within the fresh food compartment 120. Each shelf 115 may be secured to the walls 122 within the fresh food compartment 120. One or more drawers 200 may be slidably secured to the shelves 115, the internal walls 122, or to another surface within the fresh food compartment 120. In various embodiments, each of the one or more drawers 200 may be slidably secured via tracks or rails to guide the drawer 200 from a stowed position to an open position. One or more of the drawers 200 may be either a pantry drawer 205 or a crisper compartment 210, and the location and sizing of pantry drawers 205 and crisper compartments 210 shown in FIG. 2 is not intended to be limiting, and other arrangements are also contemplated. A crisper compartment 210 may more specifically be a drawer defining a storage space that is kept at a desired humidity which may be different from the remainder of the fresh food compartment 120, but that is optimal for maintaining freshness of fruits and vegetables stored within the crisper drawer.

Referring again to FIGS. 1 and 2, the refrigerator 100 may have one or more doors 160, 180 that provide selective access to the fresh food compartment 120 of the refrigerator 100, where consumables may be stored. As shown, the fresh food compartment 120 doors are designated 160, and the door for the freezer compartment 130 is designated 180. The doors 160 may be configured to transition between open positions (as shown in FIG. 2) and closed positions (as shown in FIG. 1), or be partially open such that one door 160 is open while the other remains closed.

As such, as shown in FIG. 1, the doors 160 may cover an opening to the fresh food compartment 120 in the closed position. As shown in FIG. 2, the doors 160 may provide access to the fresh food compartment 120 via the opening in the open position.

In various embodiments, the refrigerator 100 may be constructed with other door structures from the French door type shown. For example, the refrigerator 100 may only have one door as opposed to two doors as illustrated. In another example, the refrigerator 100 may further include doors mounted within the doors in other embodiments to provide access to a sub-compartment (not shown) in the door. In the embodiment depicted in the FIGS, the doors 160 may be rotatably secured to the housing 110 by one or more hinges. The doors 160 may each include an exterior panel 162 and an interior panel 164 that is disposed on an internal side of the respective exterior panel 162 of each door 160. The interior panels 164 may be configured to face the fresh food compartment 120 when the doors 160 are in closed positions (see FIG. 1). The interior panel 164 may more specifically be a door liner, similar to the internal walls 122 forming the internal liner of the fresh food compartment 120. An insulating material, such as an insulating foam, may be disposed between the exterior panel 162 and interior panel 164 of each door 160 in order to reduce the heat transfer from the ambient surroundings and increase the efficiency of the refrigerator.

The doors 160 may also include storage bins 166 that are able to hold food items or containers. The storage bins 166 may be secured to the interior panels 164 of each door 160. Alternatively, the storage bins 166 may be integrally formed within or defined by the interior panels 164 of each door 160. In yet another alternative, a portion of the storage bins 166 may be secured to the interior panels 164 of each door 160, while another portion of the storage bins 166 may be integrally formed within or defined by the interior panels 164 of each door 160. The storage bins 166 may include shelves (e.g., a lower surface upon which a food item or container may rest upon) that extend from back and/or side surfaces of the interior panels 164 of each door 160.

In certain examples, although not shown in the FIGS, the refrigerator 100 may also have a water inlet that is fastened to and in fluid communication with a household water supply of potable water. Typically, the household water supply connects to a municipal water source or a well. The water inlet may be fluidly engaged with one or more of a water filter, a water reservoir, and a refrigerator water supply line. The refrigerator water supply line may include one or more nozzles and one or more valves. The refrigerator water supply line may supply water to one or more water outlets; typically one outlet for water is in the dispensing area and another to an ice tray, which may be housed in the freezer compartment 130. The refrigerator 100 may also have a control board or controller that sends electrical signals to the one or more valves when prompted by a user that water is desired or if an ice making cycle is required.

Such a controller may be part of a larger control system and may be controlled by or may cooperate with various other controllers throughout the refrigerator 100, and one or more other controllers can collectively be referred to as a controller 150 that controls various functions of the refrigerator 100 in response to inputs or signals from sensors to control functions of the refrigerator 100. Various independent controllers are also contemplated. Each controller may include a microprocessor or central processing unit (CPU) in communication with various types of computer readable storage devices or media. Computer readable storage devices or media may include volatile and nonvolatile storage in read-only memory (ROM), random-access memory (RAM), and keep-alive memory (KAM), for example. KAM is a persistent or non-volatile memory that may be used to store various operating variables while the CPU is powered down. Computer-readable storage devices or media may be implemented using any of a number of known memory devices such as PROMs (programmable read-only memory), EPROMs (electrically PROM), EEPROMs (electrically erasable PROM), flash memory, or any other electric, magnetic, optical, or combination memory devices capable of storing data, some of which represent executable instructions, used by the controller in controlling the refrigerator 100. In an example, the fresh food compartment 120 and/or the freezer compartment 130 may have a controller 150 adjust air flow in order to alter the temperature within the compartment. In another example, the controller 150 may perform a defrost cycle responsive to a contaminant gas detection. In yet another example, the controller 150 may defer a defrost cycle based on a determination that door openings have occurred sufficient to cause sufficient airing out.

A control panel 170 may be integrated into one of the fresh food compartment doors 160. The control panel 170 may include digital controls or an external display to allow users to adjust the temperature and monitor the status of the refrigerator 100. Using the control panel 170, the user may be able to interact with various functions of the refrigerator 100.

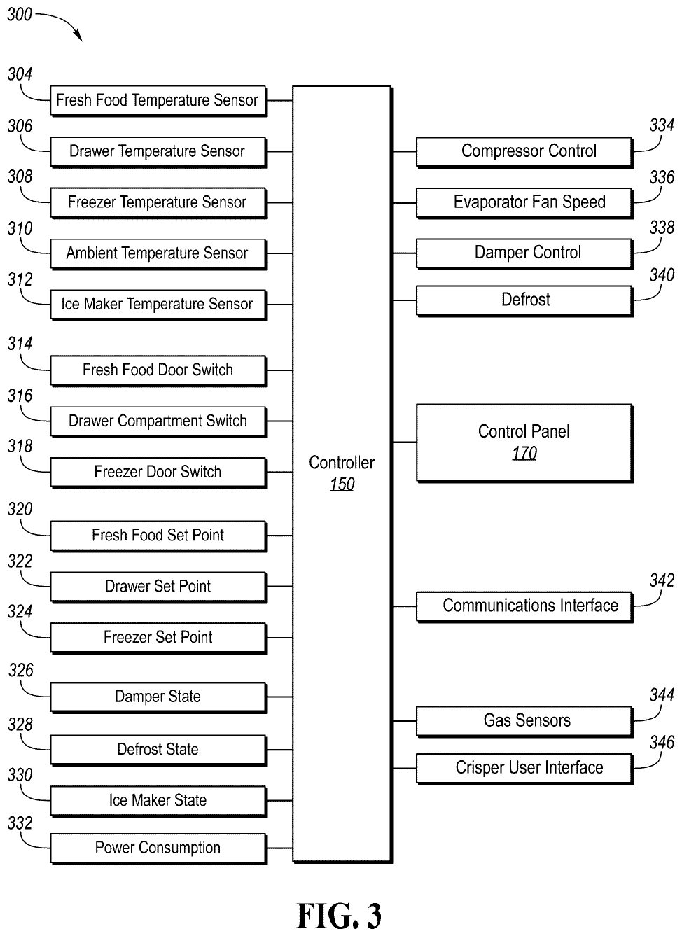

FIG. 3 illustrates a schematic view 300 of operation of the controller 150 of the refrigerator 100. Generally, the refrigerator 100 operates by circulating cool air throughout the interior using a refrigeration system that includes a compressor, condenser coils, an evaporator, and a refrigerant. The compressor pumps refrigerant between the condenser and evaporator coils. The condenser coils release heat to the outside while the evaporator coils absorb heat from the interior of the refrigerator 100, creating a cooling effect.

The controller 150 includes electronics configured to receive inputs from various sensors of the refrigerator 100 and manages the operation of the compressor, fans, and other components. Based on the sensor inputs, the controller 150 may adjust the cooling capacity of the refrigeration system and ensure that the desired temperature is maintained within the refrigerator 100.

The controller 150 may receive state information regarding a plurality of inputs. For example, the controller 150 may be electrically connected to various temperature sensors, such as a fresh food temperature sensor 304 configured to measure the temperature inside the fresh food compartment 120, a drawer temperature sensor 306 configured to measure the temperature inside the crisper compartment 210, a freezer temperature sensor 308 configured to measure the temperature inside the freezer compartment 130, an ambient temperature sensor 310 configured to measure the room temperature outside the refrigerator 100, and an ice maker temperature sensor 312 configured to measure the temperature of the ice maker, if so equipped.

The controller 150 may also receive state information from various user-movable components of the refrigerator 100. For example, the controller 150 may receive state signals from a fresh food compartment door switch 314 for monitoring whether the fresh food compartment doors 160 are open or closed, a drawer compartment switch 316 for monitoring whether a crisper compartment 210 is open or closed, a freezer compartment switch 318 for monitoring whether the freezer compartment 130 is open or closed. The controller 150 may maintain a log of the opened and closed status of the compartments over time. If one or more of the switches 314, 316, 318 are open for a predefined period of time, the controller 150 may raise an alert, in an example.

The controller 150 may also receive information indicative of the preferred temperatures to maintain in the compartments 120, 130, 210, etc. For instance, the controller 150 may receive a fresh food set point input 320 for determining the temperature at which the fresh food compartment 120 is set by a user, a drawer set point input 322 for determining the temperature at which the drawer compartment 10 is set by the user, and a freezer set point input 324 for determining the temperature at which the freezer compartment 130 is set by the user.

The controller 150 may also receive state information from other components as well. For example, the controller 150 may receive a damper state input 326 for monitoring the state of the damper. The controller 150 may also receive a defrost state input 328 for monitoring the state of the defrost. The controller 150 may also receive an ice maker state input 330 for monitoring the state of the ice maker. The controller 150 may also receive a power consumption input 332 for monitoring the power consumption of the various components of the refrigerator 100. The power consumption input 332 may be measured using a built-in power meter or may be estimated based on the current compressor state of the refrigerator 100 in an example.

Based on the received inputs, the controller 150 is configured to control various aspects of the operation of the refrigerator 100. For instance, the controller 150 may provide a compressor control 334 signal configured to control the operation of the compressor, an evaporator fan signal 336 for controlling the speed of the evaporator fan, a damper control signal 338 for controlling the operation of one or more dampers, and a defrost signal 340 for controlling one or more heaters for defrosting components where icing or condensation is not desired.

The controller 150 may also include a communications interface 342. The communications interface 342 may provide for wired and/or wireless communications between the controller 150 and external devices. For instance, the communications interface 342 may support wireless connections over protocols such as Wi-Fi, cellular, or BLUETOOTH, or wired connections such as via universal serial bus (USB) or Ethernet. The communications interface 342 may be used for various purposes, such as transferring information off the controller 150 to an external device, or receiving information from an external device to the controller 150.

The controller 150 may also be configured to receive input from one or more gas sensors 344. The gas sensor 344 may be configured to measure the concentration of gas in the air within proximity of the gas sensor 344. In one example, a gas sensor 344 may be placed in the main refrigerator cavity to measure the gas concentration in the main refrigerated food compartment area of the refrigerator 100. In another example, a gas sensor 344 may be placed in a proximity to the crisper compartment 210 to measure the gas concentration in the crisper compartment 210. In an example, the gas sensors 344 may include MQ3 sensors or ethylene sensors.

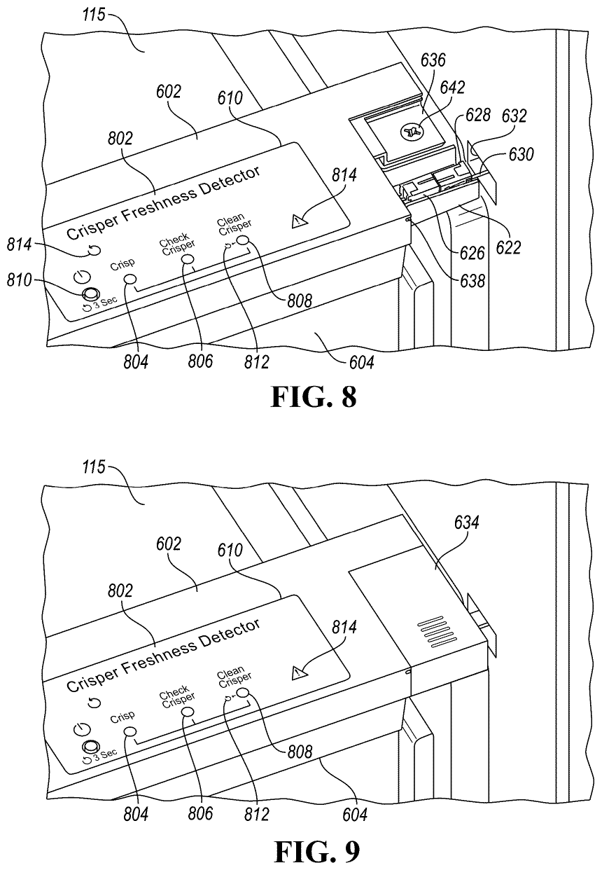

The controller 150 may also be configured to control a crisper user interface 346. In addition to the control panel 170, the refrigerator 100 may include additional controls or other user interface elements in proximity to the crispers. This crisper user interface 346 may allow the user to set options for the crisper, such as airflow level or desired temperature. The crisper user interface 346 may also display various useful information, such as current crisper temperature, and/or level of ripeness that is detected for the crisper using the gas sensor 344. Further details of an example of the crisper user interface 346 are shown in detail at least in FIG. 8.

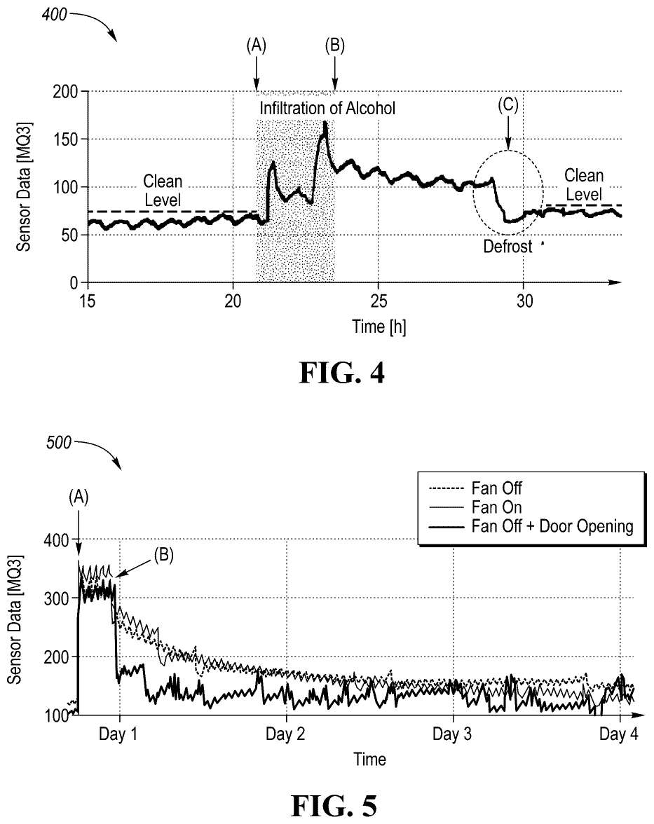

FIG. 4 illustrates an example graph 400 of sensor data 402 received from the gas sensor 344 over time in view of a defrost cycle. The sensor data 402 may be received to the controller 150 for processing, in an example. In the specific example shown, the gas sensor 344 is an MQ3 sensor, and the values of the sensor data 402 are shown in the Y-axis over time on the X-axis. The graph 400 begins in time with sensor data 402 levels that indicate a clean condition in the food compartment. At time (A), the sensor data 402 indicates an increased level of MQ3, which may be indicative of a ripeness issue. The food item causing the increased level of MQ3 may be removed at time (B). It can be seen, however, that the level of MQ3 remains elevated. Later, at time (C), a defrost cycle is run by the refrigerator 100. As can be seen, the defrost cycle reduces the level of MQ3. After the defrost cycle, the level has returned to a level indicative of a clean food compartment. Thus, performing the defrost cycle resets the air in the food compartment, allowing for the sensor data 402 received from the gas sensor 344 to be relied upon for ripeness determination.

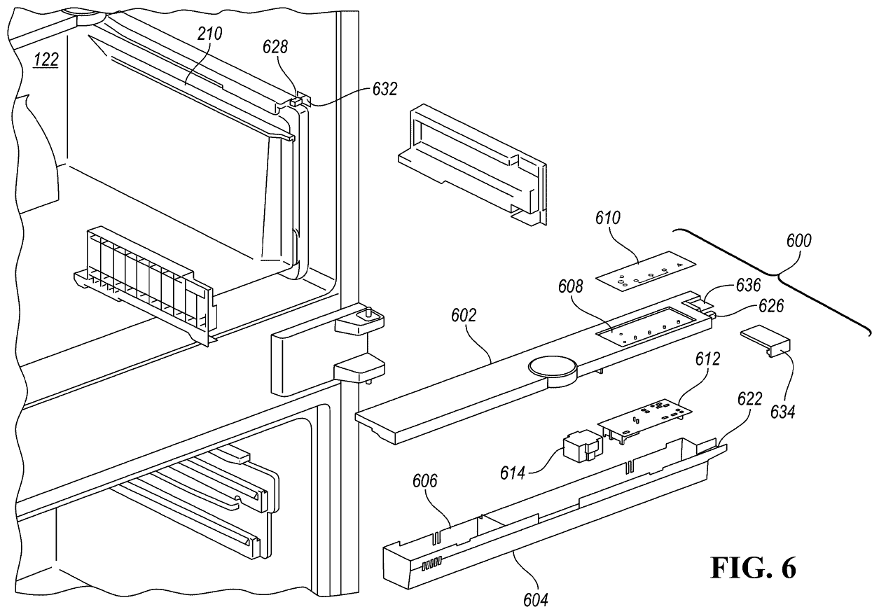

FIG. 5 illustrates an example graph 500 of sensor data 402 received from the gas sensor 344 over time in view of a door opening. The sensor data 402 from the gas sensor 344 and the state of the door switches 314, 416, 318 may be received to the controller 150 for processing, in an example. As shown, at time (A), a food item with a ripeness issue is placed into the food compartment, and at time (B), the food item with the ripeness issue is removed from the food compartment. Here again, the values from the gas sensor 344 are shown in the Y-axis over time on the X-axis. These values are shown for three scenarios—a first scenario with the fan off, a second scenario with the fan on, and a third scenario with the fan off and with a door opening event. In this example the door opening event includes opening the food compartment for at least a predefined period of time, such as five minutes in one non-limiting example.

As can be seen, the fan on and fan off scenarios are similar in terms of sensor data 402 levels. In either case, the level of sensor data 402 returns to a normal, clean level, starting at a faster rate with the rate slowing over time. However, the third scenario with the fan off and with a door opening event shows much lower levels of sensor data 402 quicker than the first and second scenarios. Thus, as with the defrost cycle, the door opening event also serves to quickly return the levels of gas in the food compartment back to normal levels.

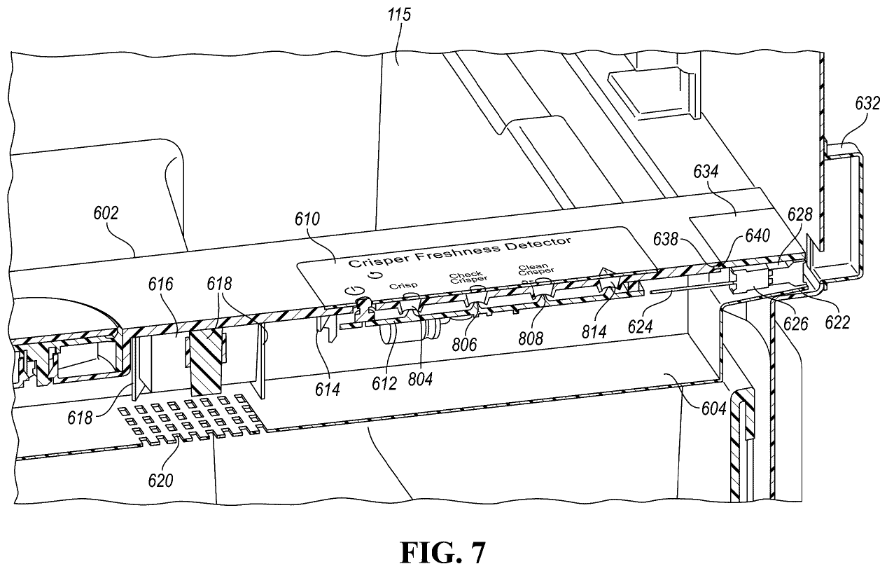

FIGS. 6-9 collectively illustrate a shelf frame sensor housing 600 accommodating the crisper user interface 346 and the gas sensor 344. FIG. 6 illustrates an example exploded perspective view of components of the shelf frame sensor housing 600 and refrigerator 100. FIG. 7 illustrates a cutaway perspective view of the shelf frame sensor housing 600 in an assembled state, showing the placement of certain internal components. FIG. 8 illustrates a close-up perspective view detailing the crisper user interface 346 and the wire connection of the shelf frame sensor housing 600 to the refrigerator 100. FIG. 9 a close-up perspective view of the wire connection of the shelf frame sensor housing 600 with the wire connection covered.

Generally, the shelf frame sensor housing 600 may be incorporated into the front trim edge of the shelf 115 above the crisper compartment 210. The shelf frame sensor housing 600 itself may generally comprise an enclosure defined by a housing top 602 and a housing bottom 604. The rear of the housing top 602 and the housing bottom 604 collectively define an opening 606 sized to receive the shelf 115, such that when assembled, the shelf frame sensor housing 600 encloses and retains the front edge of the shelf 115 above the crisper compartment 210.

The housing top 602 defines a generally flat top surface of the shelf frame sensor housing 600, and additionally, defines a recess 608 for retaining a user interface plate 610. The user interface plate 610 includes various markings or other information explaining the functionality of the crisper user interface 346. The shelf frame sensor housing 600 may also support a crisper circuit board 612 underneath the housing top 602 below the user interface plate 610. The crisper circuit board 612 may be fastened to housing top 602 and may serve to hold the electronic components of the crisper user interface 346. As best seen in FIG. 7, the crisper circuit board 612 may be retained to the housing top 602 using one or more snap hooks 614.

The shelf frame sensor housing 600 may also be configured to retain the gas sensor 344 within a sensor enclosure 616. As best seen in FIG. 7, the sensor enclosure 616 may be retained to the housing top 602 using one or more snap hooks 618. Additionally, the housing bottom 604 may define openings 620 serving to allow airflow between the interior of the crisper compartment 210 and the gas sensor 344 (not expressly shown in FIG. 7) included in the sensor enclosure 616.

The housing bottom 604 may define a channel 622 for routing of wiring 624 between the crisper circuit board 612 and the refrigerator 100. The wiring 624 may terminate in an electrical connector 626, which may be selectively attached to a corresponding electrical connector 628 of refrigerator wiring 630 exposed through a receptacle 632 defined into the side liner of the refrigerator 100.

A lid 634 may selectively cover the channel 622 to protect the connection of the electrical connector 626 and the electrical connector 628. The lid 634 is shown as installed in FIGS. 7 and 9 and as uninstalled in FIG. 8. As best seen in FIG. 8, the housing top 602 defines a lid cutaway 636 into which the lid 634 may fit. As shown in FIG. 9, when the lid 634 is installed, the lid 634 continues the upper surface of the housing top 602 to the side liner of the refrigerator 100.

To facilitate attachment of the lid 634, the housing top 602 may define a groove 638 into the vertical contour of the lid cutaway 636, while the side and rear edges of the lid 634 may define a corresponding tongue 640 to slide into and along the groove 638. When the lid 634 is removed, a fastener 642 may also be installed to secure the shelf frame sensor housing 600 to the shelf 115.

To install the shelf 115, the shelf 115 may be inserted into the refrigerator 100, the electrical connector 626 of the shelf frame sensor housing 600 may be connected to the electrical connector 628 of the refrigerator 100, and the lid 634 may be slidably attached to the housing top 602, covering the exposed connectors 626, 628. If it is desired to remove the shelf 115, such as for cleaning, then the lid 634 may be slidably detached from the housing top 602, the electrical connector 626 and the electrical connector 628 disconnected, and the shelf 115 may be removed, as it is now free of the refrigerator wiring 630.

As best seen in FIG. 8, the crisper user interface 346 includes a plurality of controls for the interaction of the user with the functionality of the intelligent crisper. Thus, the crisper user interface 346 may allow the customer to manage the operation of the gas sensor 344 in evaluating the level of ripeness of food items in the crisper compartment 210.

The controller 150 may receive inputs from the crisper user interface 346. The controller 105 may also provide status information to the crisper user interface 346. As shown, the crisper user interface 346 includes a static indication 802 that the crisper user interface 346 provides crisper freshness detection. The crisper user interface 346 may also include a crisp status indicator 804, a check crisper indicator 806, and a clean crisper indicator 808. The crisp status indicator 804 may be illuminated to indicate that the associated food compartment has no ripeness issues. The check crisper indicator 806 may be illuminated to indicate that the associated food compartment should be checked for a ripeness issue. The clean crisper indicator 808 may be illuminated to indicate that the associated food compartment should be cleaned due to detection of a ripeness issue.

The crisper user interface 346 may also include a clear crisper button 810 that, when selected, causes a manual airing out cycling of the air in the associated food compartment. The clean crisper indicator 808 may also include a graphic or other indication 812 to show that the user should press clear crisper button 810. During the manual airing out cycle, an air cleaning indicator 814 may be illuminated. A warning indicator 816 may also be illuminated when the crisper user interface 346 requests the user's attention, such as when an indication of spoilage of food items is detected in the crisper compartment 210 by the gas sensor 344.

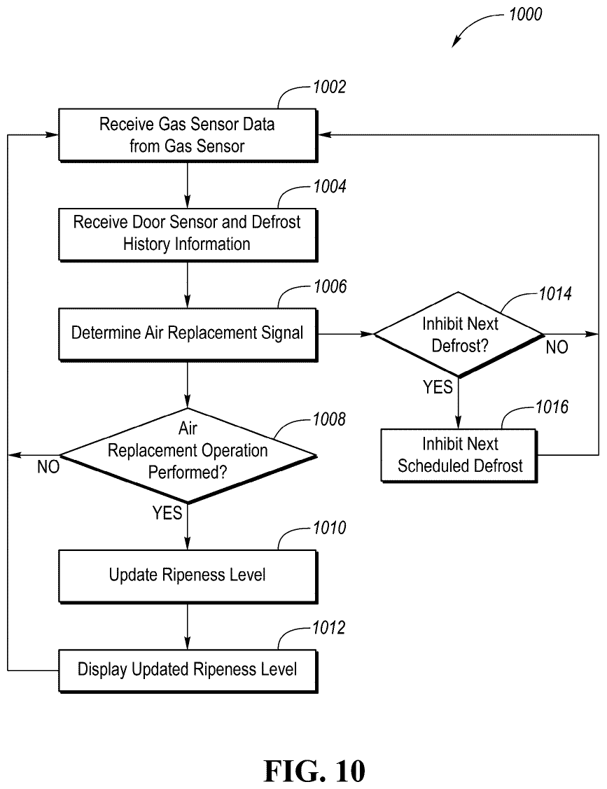

FIG. 10 illustrates an example process 1000 for the operation of the refrigerator 100 for implementing the intelligent crisper feature. In an example, the process 1000 may be performed by the controller 150 or one or more other controllers of the refrigerator 100.

At operation 1002, the refrigerator 100 receives gas sensor data 402 from the gas sensor 344. The gas sensor data 402 may be indicative of detectable gases emitted from food items stored in a food compartment. In a specific example of a gas sensor 344, the gas sensor 344 is an MQ3 sensor, and the values of the sensor data 402 are values indicative of the concentration of alcohols in the air. In a specific example of placement of the gas sensor 344, the gas sensor 344 may be integrated into a shelf frame sensor housing 600 to retain the gas sensor 344 in proximity to a crisper compartment 210, causing the gas sensor data 402 to be indicative of the levels of gases in the crisper compartment 210.

At operation 1004, the refrigerator 100 receives door sensor and defrost history information. In an example, the controller 150 may receive information indicative of when the last defrost cycle was performed by the refrigerator 100. For instance, the controller 105 may schedule defrost cycles to occur periodically, such as hourly or every other hour. The controller 150 may compute when the last defrost cycle was performed based on the period of automatic scheduling of the defrost cycles.

Also, the state of the door switches 314, 416, 318 may be received to the controller 150, which may record, store, and/or retain a history of operation of the doors to the refrigerator 100. This history may indicate information such as the timing of openings and closings of the doors. Based on the history, the controller 150 may determine the quantity of door openings that were performed since the last defrost cycle (or within another relevant period of time such as within the last hour). Additionally or alternatively, the controller 150 may determine the amount of time that the doors were opened since the last defrost cycle (or within another relevant period of time such as within the last hour). Additionally or alternatively, the controller 150 may determine whether or not any manual air cycling was performed by the customer using the clear crisper button 810.

At operation 1006, the refrigerator 100 determines an air replacement signal. This air replacement signal may be set to a value indicative of whether or not an air replacement operation was performed in the compartment. The air replacement may have been performed in different ways. In one example, the air replacement may have been performed due to the occurrence of a defrost cycle. In another example, the air replacement may have been performed responsive to the occurrence of the quantity of door openings since the last defrost cycle. In yet another example, the air replacement may have been performed manually by the user, such as responsive to the user selecting the clear crisper button 810.

At operation 1010, the refrigerator 100 determines whether to update the ripeness level based on whether an air replacement was performed. As noted herein, various sources of noise may reduce the accuracy of a determination of ripeness level. These sources of noise may include perfumes, cleaning products, etc. that provide gas emissions that are detectable by the gas sensor 224 and/or remaining gas that is present after a ripe food item has been removed. Thus, the controller 150 may ensure that air in the food compartment of the refrigerator 100 is at least partially replaced before gas sensor data 402 from the gas sensor 224 is used. This may allow for the decision-making to be performed with greater accuracy. If the air replacement signal determined at operation 1006 is set to a value indicative of occurrence of an air replacement operation, control proceeds to operation 1010. If not, control returns to operation 1002.

At operation 1010, the refrigerator 100 updates the ripeness level for the food compartment. The update may be performed because, as the air replacement was performed, the conditions in the food compartment are such that the gas sensor data 402 from the gas sensor 344 may be used as an input into the ripeness algorithm. The ripeness algorithm may determine the level of ripeness of food items in the food compartment based on the level of gas indicated in the gas sensor data 402.

At operation 1012, the refrigerator 100 displays the updated ripeness level. In an example, the ripeness level may be displayed to the crisper user interface 346. For instance, if the ripeness level indicates a first level of ripeness such that food items in the compartment are likely not spoiled, the crisp status indicator 804 may be illuminated to indicate that the associated food compartment has no ripeness issues. Or, if the ripeness level indicates a higher level of ripeness such that food items in the compartment may possibly be spoiled, the check crisper indicator 806 may be illuminated to indicate that the associated food compartment should be checked for a ripeness issue. Or, if the ripeness level indicates an even higher level of ripeness such that food items in the food compartment are likely spoiled, the clean crisper indicator 808 may be illuminated to indicate that the associated food compartment should be cleaned due to detection of a ripeness issue.

Additionally or alternatively, the ripeness level information may be displayed to the control panel 170. For instance, similar to as discussed with respect to the crisper user interface 346, different levels of ripeness for one, more than one, and/or each of the compartments 120, 130, 210, etc. may be displayed to the control panel 170. This may allow the user to understand if there are spoiled or potentially spoiled items in each of the different compartments 120, 130, 210, etc. of the refrigerator 100. After operation 1012, the process 1000 returns to operation 1002.

At operation 1014, also responsive to the determination of the air replacement signal at operation 1006, the refrigerator 100 determines whether to defer a next defrost cycle. In an example, if an air replacement was performed since the last defrost cycle, such as responsive to the occurrence of the quantity of door openings and/or was performed manually by the user, then the next defrost cycle may be inhibited by the controller 150. This may be done, for example, because the door openings and/or manual cycle may have served to perform the purpose of melting any accumulated frost that may have formed on the cooling system. If, however, the last air replacement was due to a defrost cycle, then the next scheduled periodic defrost cycle should still be performed.

If the next defrost cycle is being inhibited, control passes to operation 1016 to cause the controller to set to avoid performance of the next defrost cycle. Accordingly, at operation 1016, the refrigerator 100 does not perform the next scheduled periodic defrost cycle, or in other examples, delays the performance of the next defrost cycle. After operation 1016, control may also return to operation 1002.

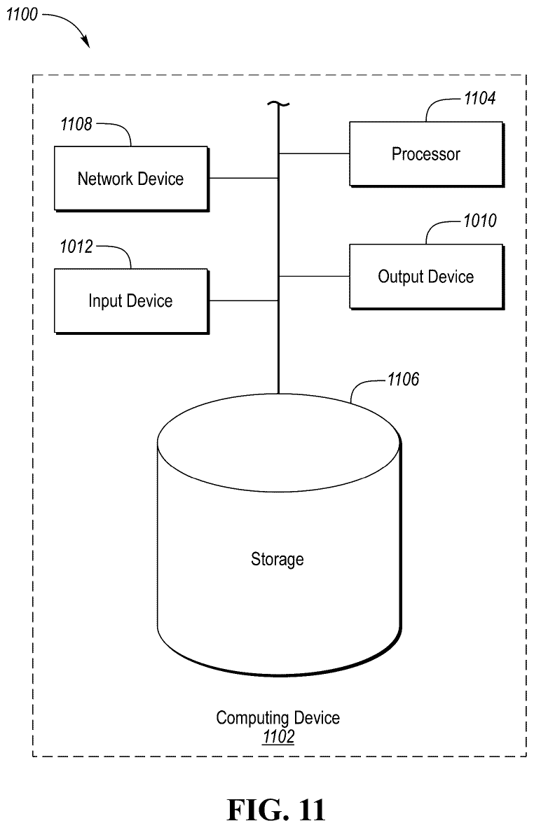

FIG. 11 illustrates an example 1100 of a computing device 1102 for implementing issue reporting functionality. Referring to FIG. 11, and with reference to FIGS. 1-10, the controller 150 may be an example of such a computing device 1102. As shown, the computing device 1102 may include a processor 1104 that is operatively connected to a storage 1106, a network device 1108, an output device 1110, and an input device 1112. It should be noted that this is merely an example, and computing devices 1102 with more, fewer, or different components may be used.

The processor 1104 may include one or more integrated circuits that implement the functionality of a CPU and/or graphics processing unit (GPU). In some examples, the processors 1104 are a system on a chip (SoC) that integrates the functionality of the CPU and GPU. The SoC may optionally include other components such as, for example, the storage 1106 and the network device 1108 into a single integrated device. In other examples, the CPU and GPU are connected to each other via a peripheral connection device such as peripheral component interconnect (PCI) express or another suitable peripheral data connection. In one example, the CPU is a commercially available central processing device that implements an instruction set such as one of the x86, ARM, Power, or microprocessor without interlocked pipeline stages (MIPS) instruction set families.

Regardless of the specifics, during operation the processor 1104 executes stored program instructions that are retrieved from the storage 1106. The stored program instructions, accordingly, include software that controls the operation of the processors 1104 to perform the operations described herein. The storage 1106 may include both non-volatile memory and volatile memory devices. The non-volatile memory includes solid-state memories, such as not AND (NAND) flash memory, magnetic and optical storage media, or any other suitable data storage device that retains data when the system is deactivated or loses electrical power. The volatile memory includes static and dynamic RAM that stores program instructions and data during operation of the refrigerator 100.

The GPU may include hardware and software for display of at least two-dimensional (2D) and optionally three-dimensional (3D) graphics to the output device 1110. The output device 1110 may include a graphical or visual display device, such as an electronic display screen, projector, printer, or any other suitable device that reproduces a graphical display. As another example, the output device 1110 may include an audio device, such as a loudspeaker or headphone. As yet a further example, the output device 1110 may include a tactile device, such as a mechanically raisable device that may, in an example, be configured to display braille or another physical output that may be touched to provide information to a user.

The input device 1112 may include any of various devices that enable the computing device 1102 to receive control input from users. Examples of suitable devices that receive human interface inputs may include keyboards, mice, trackballs, touchscreens, voice input devices, graphics tablets, and the like.

The network devices 1108 may each include any of various devices that enable the refrigerator 100 to send and/or receive data from external devices over networks. Examples of suitable network devices 1108 include an Ethernet interface, a Wi-Fi transceiver, a cellular transceiver, or a BLUETOOTH or BLUETOOTH low energy (BLE) transceiver, or other network adapter or peripheral interconnection device that receives data from another computer or external data storage device, which can be useful for receiving large sets of data in an efficient manner.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to strength, durability, life cycle, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A method for performing spoiled food detection by a refrigerator, comprising:
receiving gas sensor data from a gas sensor, the gas sensor data being indicative of detectable gases emitted from food items stored in a food compartment;
responsive to completion of an air replacement operation in the food compartment, using the gas sensor data to determine whether spoiled food items are detected in the food compartment; and
displaying, to a user interface, an indication of a level of spoilage of food items detected in the food compartment.

2. The method of claim 1, wherein the air replacement operation is a defrost cycle.

3. The method of claim 2, further comprising:
scheduling the defrost cycle; and
checking for presence of spoiled food items based on the gas sensor data, responsive to completion of the scheduled defrost cycle.

4. The method of claim 3, wherein the defrost cycle is scheduled periodically.

5. The method of claim 1, wherein the air replacement operation includes a predefined amount of door openings.

6. The method of claim 5, further comprising:
recording a history of operation of a door to the refrigerator, the history indicating timing of openings and closings of the door.

7. The method of claim 6, further comprising:
responsive to the history indicating at least the predefined amount of door openings open since a last defrost cycle, inhibiting a next defrost cycle.

8. The method of claim 7, further comprising:
checking for presence of spoiled food items based on the gas sensor data, despite the inhibiting of the next defrost cycle, due to the door openings clearing air in the food compartment to facilitate measurement of the emitted gases.

9. The method of claim 6, wherein the amount of door openings includes a total amount of time that the door is open.

10. The method of claim 6, wherein the amount of door openings includes a quantity of times that the door is opened.

11. The method of claim 1, wherein the gas sensor is embedded in a shelf frame housing.

12. The method of claim 11, wherein the shelf frame housing further includes the user interface.

13. A refrigerator implementing spoiled food detection, comprising:
a shelf frame sensor housing comprising a user interface and a gas sensor configured to provide gas sensor data indicative of gases emitted from food items stored in the refrigerator; and
one or more controllers configured to:
responsive to completion of an air replacement operation in the refrigerator, use a gas signal from the gas sensor to determine whether spoiled food items are detected in the refrigerator, and
display, to the user interface, an indication of a level of spoilage of food items detected in the refrigerator.

14. The refrigerator of claim 13, wherein the air replacement operation is a defrost cycle performed by the refrigerator.

15. The refrigerator of claim 14, wherein the one or more controllers are further configured to:
schedule the defrost cycle; and
check for presence of spoiled food items based on the gas signal, responsive to completion of the scheduled defrost cycle.

16. The refrigerator of claim 15, wherein the one or more controllers are further configured to schedule the defrost cycle on a periodic timeframe.

17. The refrigerator of claim 13, wherein the air replacement operation includes a predefined amount of door openings.

18. The refrigerator of claim 17, wherein the one or more controllers are further configured to:
record a history of operation of a door to the refrigerator, the history indicating timing of openings and closings of the door.

19. The refrigerator of claim 18, wherein the one or more controllers are further configured to:
responsive to the history indicating at least the predefined amount of door openings open since a last defrost cycle, inhibit a next defrost cycle.

20. The refrigerator of claim 19, wherein the one or more controllers are further configured to:
check for presence of spoiled food items based on the gas sensor data, despite the inhibiting of the next defrost cycle, due to the door openings clearing air in the refrigerator to facilitate measurement of the emitted gases.

21. The refrigerator of claim 18, wherein the amount of door openings includes a total amount of time that the door is open.

22. The refrigerator of claim 18, wherein the amount of door openings includes a quantity of times that the door is opened.

23. The refrigerator of claim 13, wherein the gas sensor is embedded in a shelf frame housing of a shelf of the refrigerator.

24. The refrigerator of claim 23, wherein the shelf frame housing further includes the user interface.
